(19) United States
(12) Patent Application Publication
(10) Pub. No.: US 2021/0121636 A1
(43) Pub. Date: Apr. 29, 2021

(54) AUTO DISABLE PREFILLED SYRINGE WITH PLUNGER ROD SPRING

(71) Applicant: KRUI Medical AB, Helsingborg (SE)

(72) Inventor: Per KNUTSSON, Helsingborg (SE)

(73) Assignee: KRUI Medical AB, Helsingborg (SE)

(21) Appl. No.: 17/044,135

(22) PCT Filed: Mar. 21, 2019
(86) PCT No.: PCT/EP2019/057103

§ 371 Date: Sep. 30, 2020

(30) Foreign Application Priority Data
Apr. 6, 2018 (SE) 1850388-8

(51) Int. Cl. A61M 5/315

(52) U.S. Cl. CPC A61M 5/31511; A61M 2005/31508; A61M 5/31578; A61M 5/31501

(57) ABSTRACT

The present invention discloses a plunger rod (104) for use in connection with an injection syringe barrel (102). The plunger rod (104) comprises an elongated rod (114). The elongated rod (114) comprises a proximal end (107) adapted to receive an applied force along an axis (1000). It further comprises a distal end (105) adapted to transmit the applied force along the axis (1000). The plunger rod (104) is adapted to be inserted into a barrel (102) of a syringe (100) extending along the axis (1000). The plunger rod (104) is further adapted to transmit force along the axis (1000) to a plunger stopper (103) disposed within the barrel (102). The plunger rod (104) further comprises a radial spring mechanism (106, 117, 118, 119, 120), adapted to interact with an inner surface of the barrel (102).

### TECHNICAL FIELD

The present invention relates to a prefilled syringe with a construction that prevents re-usage of the syringe and wherein the syringe may be manufactured at a cost similar to a conventional prefilled syringe.

### BACKGROUND

The business area of prefilled syringes is undergoing strong expansion. There are several reasons for this, but one of the main reasons is that the prefilled syringes are “ready for use”. Therefore, the need for the users to fill the syringes and the handling of the drugs and syringes before use is minimized. This will accordingly reduce the risk of infections and assure the quantity and the quality of the drug contained within the syringe.

Prefilled syringes are usually made of glass with highest quality. The reason for this is that the drug contained within the syringe carries the highest cost. The syringes are constructed such that they have a minimal dead space, which will assure that almost all drug or medicine contained within the syringe will be released. However, these positive characteristics of the syringes will make it tempting for users to reuse them, as conventional syringes, by refilling the syringes with drugs again. Misusage of syringes is a common issue; over 2 million users each year are infected with severe blood transmittable deceases, such as HIV, Hepatitis B and C. By current constructions and designs of prefilled syringes, this problem is not complete solved, and the attempts to solve the problem are done at a very high cost.

Hence, there is a need to provide a syringe which addresses the disadvantages and shortcomings described above and to provide a non-reusable syringe.

### SUMMARY

Accordingly, the present invention preferably seeks to mitigate, alleviate or eliminate one or more of the above-identified deficiencies in the art and disadvantages singly or in any combination and solves at least the above mentioned problems by proposing a solution according to the appended independent claims. Advantageous embodiments are defined in the appended dependent claims.

In a first of its aspects, this disclosure therefore presents a plunger rod for use in connection with an injection syringe barrel. The plunger rod comprises an elongated rod. The elongated rod comprises a proximal end adapted to receive an applied force along an axis. It further comprises a distal end adapted to transmit the applied force along the axis. The plunger rod is adapted to be inserted into a barrel of a syringe extending along the axis. The plunger rod is further adapted to transmit force along the axis to a plunger stopper disposed within the barrel. The plunger rod further comprises a radial spring mechanism, adapted to interact with an inner surface of the barrel.

One advantage of this is that the plunger rod may be manufactured separately from the syringe. Another advantage of this is that the plunger rod may be recycled or more efficiently disposed of, since it is not damaged when removed from the barrel. Another advantage of this is that since the plunger rod is not connected to the plunger stopper, the plunger stopper cannot be pulled back. An advantage of the radial spring mechanism is that the plunger rod will be stabilized and directed by the inner surface of the barrel.

In some embodiments, the plunger rod is made of a thermoplastic polymer having a Young's modulus within the interval 500 MPa to 2000 MPa.

In some embodiments, the rod of the plunger rod has a cross-shaped cross-section comprising four supporting ribs extending radially from the center of the rod. At least one supporting rib comprises the spring mechanism.

One advantage of this is that the plunger rod will have a smaller friction engaging surface and requires less material to manufacture.

In some embodiments, the four supporting ribs comprise interaction knobs.

One advantage of this is that it may be easier to optimize the applied spring force.

In some embodiments, the four supporting ribs comprise interaction discs.

One advantage of this is that it may be easier to optimize the applied spring force.

In some embodiments, the distal end of the plunger rod comprises a spring portion adapted to being displaced radially from their relaxed position once the plunger rod is inserted into said barrel of said syringe.

One advantage of this is that the spring portion allows for simple adjustment of the friction force of the supporting rib against the inner surface of the barrel.

In some embodiments, at least one of said four supporting ribs comprise spring portions adapted to being displaced radially from their relaxed position once the plunger rod is inserted into said barrel of said syringe.

One advantage of this is that the spring portion allows for simple adjustment of the friction force of the supporting rib against the inner surface of the barrel.

In some embodiments, the spring portion comprises an axial slit.

One advantage of this is that it may be easier to optimize the applied spring force.

In some embodiments, the axial slit is arranged in the middle of the rod.

One advantage of this is that it may be easier to optimize the applied spring force.

In some embodiments, the axial slit is contained within said at least one supporting rib.

One advantage of this is that there is no risk for the slit to interact with a proximal end of the barrel upon insertion of the plunger rod into the barrel.

In some embodiments, the spring portion comprises an interaction spoke.

One advantage of this is that there is a smaller risk for the interaction spoke to interact with a proximal end of the barrel upon insertion of the plunger rod into the barrel.

In some embodiments, the spring portion comprises a set of reeds.

One advantage of this is that the reeds may more easily conform with the inner surface of the barrel. There is also a smaller risk for the reeds to interact with a proximal end of the barrel upon insertion of the plunger rod into the barrel.

In a second of its aspects, this disclosure presents a syringe. The syringe comprises a barrel with a needle arranged at a distal end of the barrel. The barrel is adapted to contain a drug. The barrel has a tubular wall extending proximally from the distal end, whereby the barrel extends along an axis. The syringe further comprises a plunger stopper disposed within the barrel. The plunger stopper is adapted to be in fluid-tight engagement with the tubular wall of the barrel. The plunger stopper is displaceable along the axis within the barrel. The barrel comprises an opening arranged at the proximal end of the barrel. The opening is adapted to receive a plunger rod according to a first aspect of this disclosure extending along the axis. The plunger stopper is adapted to receive force along the axis from said plunger rod.

In some embodiments, the syringe contains the drug.

One advantage of this is that pre-filled syringes reduce risk of spillage, overdosing and contamination.

In some embodiments, the plunger stopper is made of an elastomer having a Young's modulus within the interval 25 MPa to 300 MPa.

In some embodiments, the plunger stopper is adapted to only be displaceable along the axis towards the distal end of the barrel.

One advantage of this is that it further prevents the syringe from being reused.

In some embodiments, the plunger stopper comprises a thread profile.

One advantage of this is that it allows an under pressure to be formed if necessary.

Further advantages will be apparent from the detailed description as well as the appended dependent claims.

### BRIEF DESCRIPTION OF THE DRAWINGS

These and other aspects, features and advantages of which the invention is capable, will be apparent and elucidated from the following description of non-limiting embodiments of the present invention, reference being made to the accompanying drawings, in which

FIG. 1a is a longitudinal section view of a plunger rod according to one example;

FIG. 1b is an isometric view of the same plunger rod;

FIG. 2 is a longitudinal section view of different plunger rods according to six examples;

FIG. 3 shows a longitudinal section view and an isometric view of a syringe according to one example;

FIG. 4 shows a longitudinal section view and an isometric view of a syringe according to one example;

FIG. 5 shows a longitudinal section view and an isometric view of a syringe according to one example;

FIG. 6 shows a longitudinal section view and an isometric view of a syringe according to one example;

FIG. 7a shows an isometric view of a threaded adapter according to one example;

FIG. 7b shows a longitudinal section view of a syringe according to one example;

FIG. 8a shows a top view of a packaging of a plunger rod and syringe according to one example;

FIG. 8b shows an isometric view of a packaging of a plunger rod and syringe according to one example

### DETAILED DESCRIPTION OF THE EMBODIMENTS

Embodiments of the present invention will be described in more detail below with reference to the accompanying drawings in order for those skilled in the art to be able to carry out the invention. The invention may, however, be embodied in many different forms and should not be construed as limited to the embodiments set forth herein. Rather, these embodiments are provided so that this disclosure will be thorough and complete, and will fully convey the scope of the invention to those skilled in the art. The embodiments do not limit the invention, but the invention is only limited by the appended patent claims. Furthermore, the terminology used in the detailed description of the particular embodiments illustrated in the accompanying drawings is not intended to be limiting of the invention.

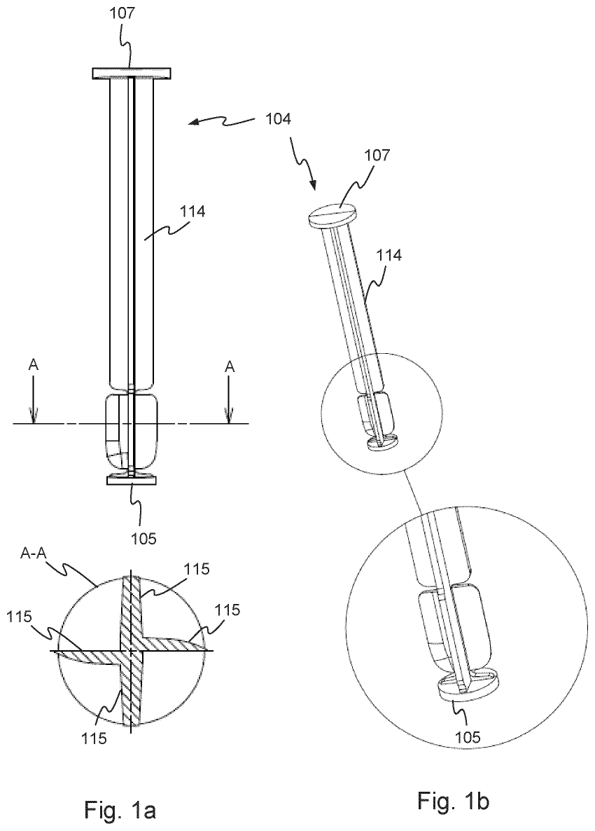

Referring to FIGS. 1a-b, one example of a plunger rod 104 according to the teachings herein is shown. The plunger rod 104 comprises an activation plate 107 at its proximal end for applying force thereto.

The plunger rod 104 further comprises a rod 114 that preferably extends at least the length of a barrel 102 of a syringe 100 that it is intended to be inserted into. The cross-section A-A of the rod 114 shown in FIG. 1a illustrates four supporting ribs 115 extending radially from the center of the rod 114. A width of the rod 114 at the cross section A-A is preferably equal to or within 10% greater than the inner diameter of the barrel 102 of the syringe 100 that the plunger rod 104 is intended to be inserted into; in order to ensure a tight fit in the barrel 102 which is used to guide the plunger rod 104. The lateral ends of supporting ribs 115 are beveled, for these supporting ribs to yield to fit snuggly with the inner surface of the barrel 102. In this way, the position of the rod 114 in the barrel 102 may be maintained when no pushing or pulling of the rod 114 in distal or proximal direction, respectively, is performed

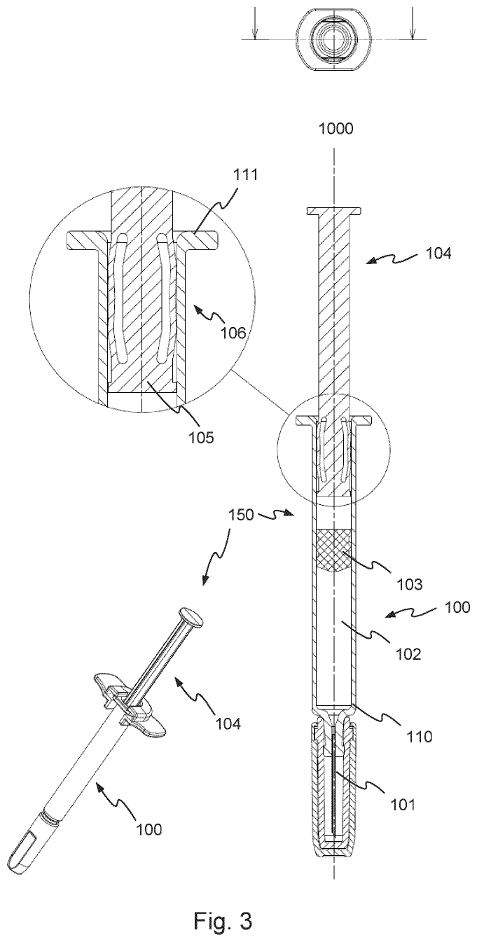

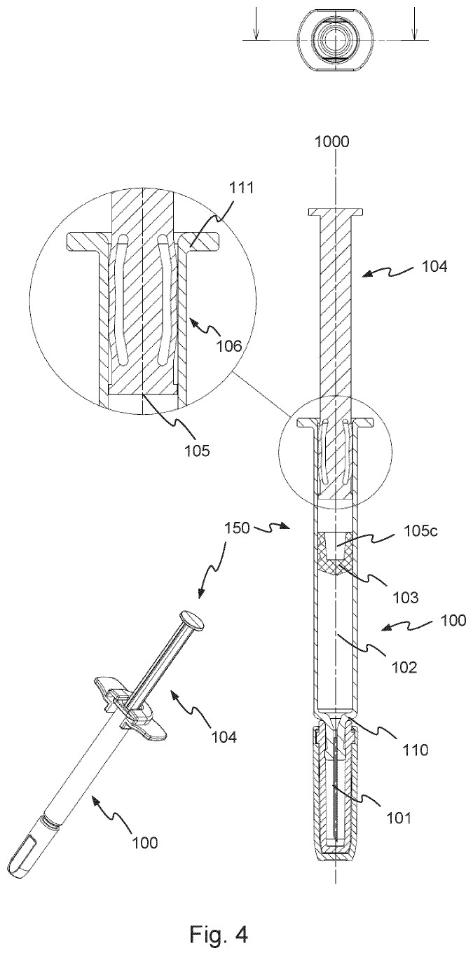

The plunger rod 104 further comprises a push plate 105 at its distal end 105, which is used to push a plunger stopper 103, as shown in FIGS. 3 and 4, in the barrel 102 toward a distal end 110 of the barrel 102 after a force is applied to the proximal end 107 of the plunger rod 104.

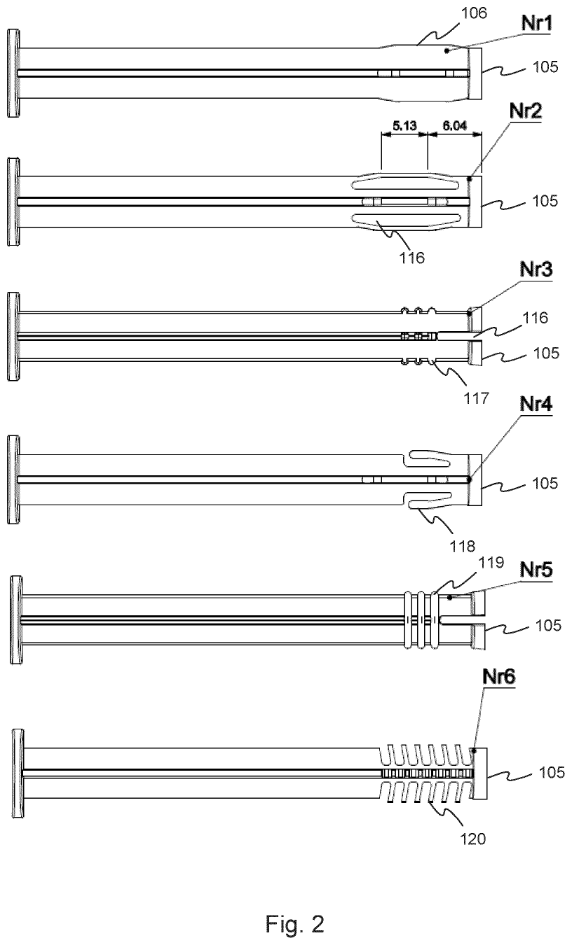

With reference to FIG. 2, six examples of a plunger rod 104 according to the teachings herein are shown. Each example has a different non-limiting embodiment of a spring portion 106 at the distal end 105 of the plunger rod 104.

Embodiment Nr1 uses press fitting to insert a distal end 105 of the plunger rod 104 with two opposing supporting ribs 115 having a diameter larger than the inner diameter of the barrel 102 of the syringe 100. This achieves a radial deformation of the supporting ribs 115 which will radially apply a spring force to the barrel 102, thereby achieving a spring portion 106. By changing the size of portions of the supporting ribs 115, the applied spring force may be optimized to be large enough to ensure the plunger rod 104 sits tight against the barrel 102 of the syringe 100 and small enough to ensure that the operation of the syringe 100, meaning the transfer of force from the plunger rod 104 to the plunger stopper 103 leading to a distal displacement, is not disturbed.

Embodiment Nr2 uses a similar radial extension of two opposing supporting ribs 115 in order to achieve a spring portion 106 at the distal end 105 of the plunger rod 104. However, in this case the spring portion 106 comprises an axial slit 116. An advantage of this is that it may be easier to optimize the applied spring force according to the limits above. The axial slit 116 is contained within the ribs 115, such that the slit 116 does not open up into the periphery of the rod 114. In this way, there is no risk for the slit to interact with a proximal end 111 of the barrel 102 upon insertion of the plunger rod 104 into the barrel 102. If the slit 116 opened up into the periphery of the rod 114, there would be a risk that a protrusion formed laterally of the slit 116, pointing distally, would be deformed during demoulding to further increase the risk for the slit 116 to interact with the proximal end 111 of the barrel upon insertion of the plunger rod 104.

Embodiment Nr3 uses a similar concept as the previous embodiment of a radial extension combined with an axial slit opening up into the push plate 105 to achieve a spring portion 106. In this case the axial slit is placed in the middle of the rod 114, separating the supporting ribs 115 from each other. In this embodiment the width of the push plate 105 is somewhat larger than the inner diameter of the barrel 102 in relaxed state, such that the periphery of the push plate 105 will interact with the inner surface of the barrel 102, in compressed state to maintain the plunger rod 104 therein also when no pushing or pulling is performed. An advantage of this is that it may be easier to optimize the applied spring force according to the limits above. Supporting ribs 115 are provided with interaction knobs 117. The interaction knobs 117 will, during insertion of the plunger rod 104 into the barrel 102 interact with the inner surface of the barrel 102 in a retaining manner, whilst simultaneously allowing for distal and proximal movement of the plunger rod 104 upon pushing and pulling, respectively, of the plunger rod 104.

Embodiment Nr4 is most similar to embodiment Nr2, except that the slit 116 has been extended radially in the proximal direction of the plunger rod 104, forming an interaction spoke 118 extending substantially proximally and somewhat radially from the supporting ribs 115. Hence, the slit 116 opens up into the periphery of the rod 114 in the proximal direction, such that the spokes 118 anyhow will yield centrally when inserting the plunger rod 104 into the barrel 102, without an increased risk of having the slit 116 interacting with the proximal end 111 of the barrel 102. An advantage of this is that it may be easier to optimize the applied spring force according to the limits above.

Embodiment Nr5 is most similar to embodiment Nr3, except that the supporting ribs 115 are reinforced with interaction discs 119 instead of interaction knobs 117. The interaction discs 119 connect the ribs 115 and are dimensioned and configured to interact with the inner surface of the barrel 102 when the plunger rod 104 is displaced within the barrel 102. The interaction discs 119 will, during insertion of the plunger rod 104 into the barrel 102 interact with the inner surface of the barrel 102 in a retaining manner, whilst simultaneously allowing for distal and proximal movement of the plunger rod 104 upon pushing and pulling, respectively, of the plunger rod 104. An advantage of this is that it may be easier to optimize the applied spring force according to the limits above.

Embodiment Nr6 is most similar to embodiment Nr4, except that instead of a spoke 118 the ribs are provided with a set of reeds 120. The reeds 120 are thinner than the spokes 118, and will conform with the inner surface of the barrel 102. The reeds 120 are directed substantially radially and somewhat proximally. An advantage of this is that it may be easier to optimize the applied spring force according to the limits above.

FIG. 3 illustrates one example of a syringe 100 comprising an example plunger rod 104 corresponding to embodiment Nr2 from FIG. 2. As the plunger rod 104 is inserted into a proximal end 111 of a barrel 102 axially along an axis 1000, the spring portion 106 at the distal end 105 of the plunger rod 104 is displaced and a spring force is applied radially to the barrel 102.

The syringe 100 comprises a barrel 102 with a needle 101 attached to a distal end 110 of the barrel 102. The needle 101 is mechanically and hermetically attached to the syringe as known in the art, such as molded or glued, around the rear end of a hollow needle, whereby liquid passage is allowed in both directions, from the rear end of the needle unit to and through a needle tip of the hollow needle. The needle 101 is usually very thin, with a surface diameter of 0.3-0.6 mm. The barrel 102 of the syringe 100 may be made of any material suitable for containing a drug, but is preferably made of glass. The glass is preferably transparent. The barrel 102 preferably comprises a proximal end 111 which is adapted to act as a counter-balance to the force applied to the proximal end 107 of the plunger rod 104. The barrel 102 is adapted to contain a drug. The drug may be in liquid form to be injected into a patient or user. The barrel 102 has a distal end 110, to which the needle 101 is attached, and a tubular wall extending proximally from the distal end. The syringe 100 extends along an axis 1000 which defines the direction in which the barrel 102 extends, e.g. the barrel 102 extends along the axis 1000.

A plunger stopper 103 is disposed within the barrel 102. The plunger stopper 103 is adapted to be in fluid-tight engagement with the tubular wall of the barrel 102. By being in fluid-tight engagement with the tubular wall of the barrel 102, the drug contained within the barrel 102 is prevented from leaking out of the syringe 100 in any other direction than through the needle 101 at the distal end 110 of the barrel 102. The plunger stopper 103 is disposed within the barrel 103 after the syringe 100 has been filled with the drug.

The plunger stopper 103 may however regardless of the thread profile 105b be adapted to receive force along the axis 1000 from a plunger rod 104 without a thread profile 105a. By displacing the plunger stopper 103 and the plunger rod 104 along the axis 1000 in a direction within the barrel 102 towards the distal end 110 with the attached needle 101, injection of the drug is achieved. When the plunger stopper 103 and the plunger rod 104 have been moved as far as possible towards the distal end 110 of the barrel 102, almost all drug contained within the syringe 100 have been released and injected to the user. Since the syringe 100 is constructed such that dead space is minimized, almost all of the valuable drug will be released. After the drug has been injected, the plunger rod 104 may be discarded separate from the syringe 100. This is advantageous as used syringes need to be disposed of in special containers, which need to be handled in a safe and costly manner. Therefore, by separating the plunger rod 104, it will no longer take up unnecessary space in these containers and also be more efficiently recycled. In one embodiment according to the teachings herein, the syringe 100 contains the drug. The drug may need to be refrigerated, which means that being able to pack the syringes 100 efficiently is even more important. This is yet another advantage of being able to keep the plunger rod 104 separate.

FIG. 4 illustrates another example of a syringe 100 comprising an example plunger rod 104. In this embodiment, the plunger stopper 103 has a hollow portion 105c. As long as the proximal end of the plunger stopper 103 may receive the transmitted force of the plunger rod 104, the plunger stopper 103 is within the scope of the amended claims. Thusly, the center of the plunger stopper 103 may be hollow to conserve materials or, as seen in FIG. 4, the proximal end of the plunger stopper 103 may only comprise an outer ridge.

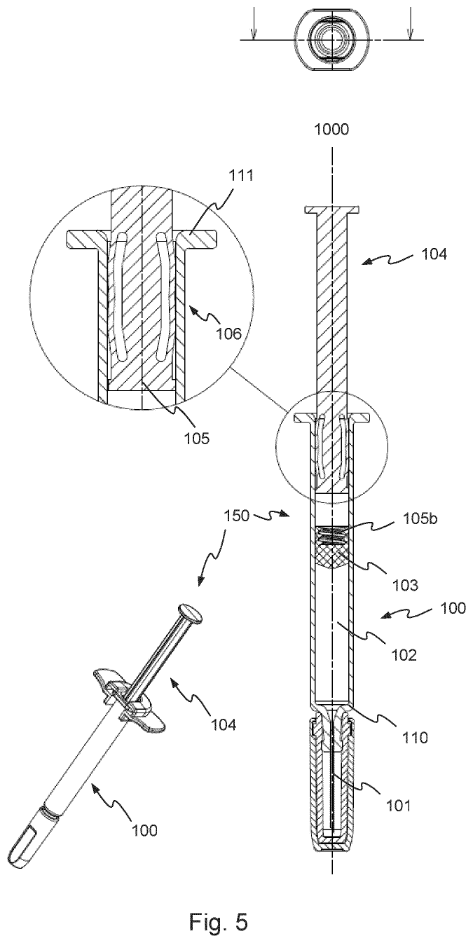

FIG. 5 illustrates another example of a syringe 100 comprising an example plunger rod 104. In this embodiment the plunger stopper 103 has a thread profile 105b which is adapted to receive a piece with a thread profile 105a that engages with the thread profile 105b of the plunger stopper 103. A thread is a helical structure used to convert between rotational and linear movement or force, and the thread is a ridge wrapped around a cylinder or cone in the form of a helix. The plunger stopper 103 according to the teachings herein is thus adapted to receive such a cone or cylinder with a thread profile 105a configured to engage with the thread profile 105b of the plunger stopper 103.

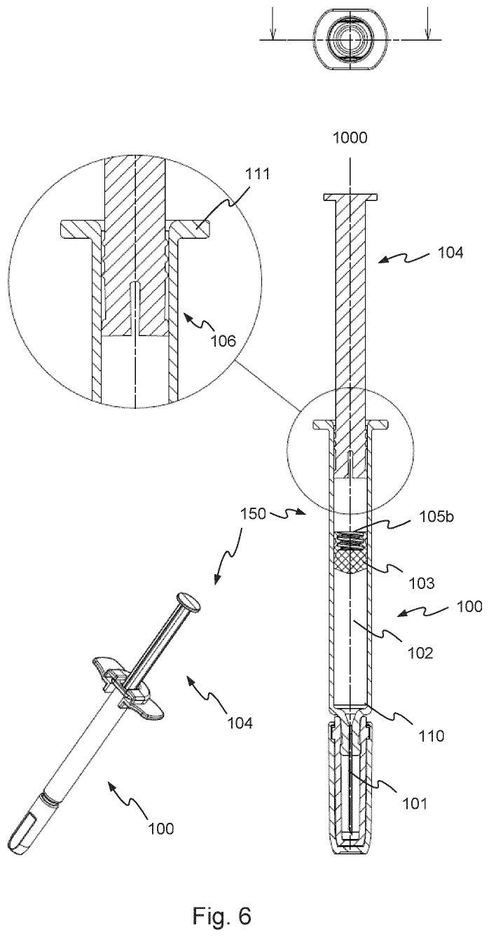

FIG. 6 illustrates another example of a syringe 100 comprising an example plunger rod 104, this time corresponding to embodiment Nr3 from FIG. 2.

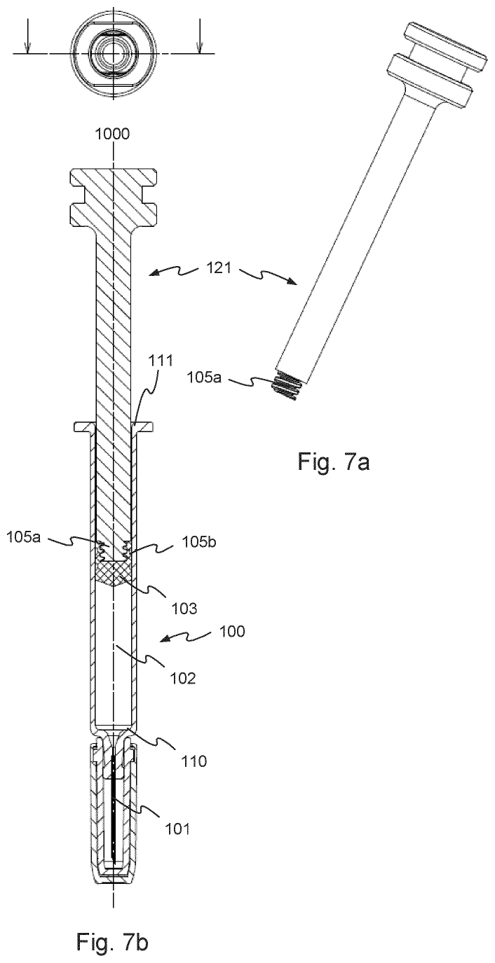

FIGS. 7a-b illustrate one example of a syringe 100 comprising an example threaded adapter 121 comprising a thread profile 105a. The thread profile 105a of the threaded adapter 121 engages with the thread profile 105b of the plunger stopper 103. Because the plunger stopper 103 and the threaded adapter 121 have engaging thread profiles 105b, 105a, the two parts may be effectively attached to each other. The plunger stopper 103 represents an external thread and the threaded adapter 121 represents an internal thread. The engaging thread profiles 105b, 105a of the plunger stopper 103 and the threaded adapter 121 work as a screw with a matching bolt. By using a fastening element, such as engaging thread profiles 105a, 105b, the fastening element is relatively easy and cheap to produce, without the need to add any additional materials or components except the plunger stopper 103 and the threaded adapter 121.

By engaging, and thereby attaching, the plunger stopper 103 and the threaded adapter 121 with each other by the engaging thread profiles 105b, 105a, the two parts may be displaceable together along the axis 1000 within said barrel 102. By displacing the plunger stopper 103 and the threaded adapter 121along the axis 1000 in a direction within the barrel 102 towards the distal end 110 with the attached needle 101, injection of the drug is achieved. The thread engagement between the threaded adapter 121 and the plunger stopper 103 may allow for a displacement long the axis 1000 in a direction within the barrel 102 towards the proximal end 111 of the barrel 102.

This may be used during manufacturing to form an under pressure which may be beneficial. It is also possible to use this threaded adapter 121 in order to make the syringe 100 reusable. The threaded adapter 121 should be integral in the assembly/filling machine.

If the need exists to create a negative pressure in the syringe 100 (aspirate), after the plunger stopper 103 has been fitted into the syringe 100. Then it is possible to equip the mounting machine with a stainless steel threaded adapter 121, which then thread in the internal thread 105b of the plunger stopper 103, which can cause a negative pressure, pulling the plunger stopper 103 backwards (aspirate), which in turn creates the negative pressure in the area where the medication is located in the syringe 100. This in turn should prevent leakage through the needle. The last step is to turn the thread rotation for the threaded adapter 121, and thread it out of the plunger stopper 103. This station is easy to integrate into the prefilled mounting machine.

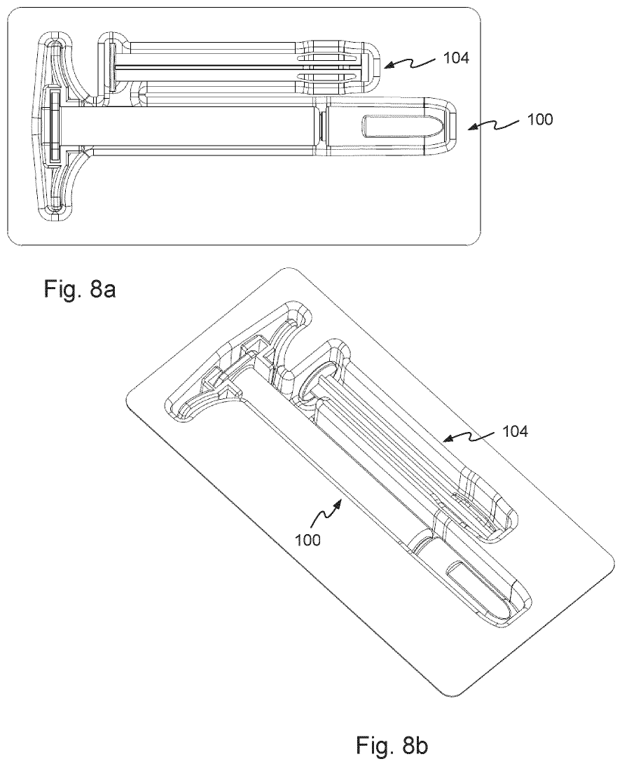

FIGS. 8a-b illustrate one example of a packaging of a plunger rod 104 and syringe 100 according to this disclosure. Because the plunger rod 104 is separate from the syringe 100, they may be packed side-by-side in an unconventional manner, as shown. This conserves space and allows for more efficient storage and shipping of the product, which is specifically asked for with regard to pre-filled syringes, since they almost exclusively are stored and transported in cold storage. Another advantage of this packaging solution is that it is less susceptible to outside forces damaging the product or accidentally injecting a portion of the drug of a pre-filled syringe 100 into its packaging. A further advantage of this packaging solution is that the center of the package is closer to the center of the syringe 100 than conventional solutions, which means that the product may be more recognizable and helps a costumer or medical professional identify a specific brand or dosage.

One advantage of the described embodiments is that the costs for the manufacturing methods used to produce the syringes according to the teachings herein will not be increased, while still assuring that the syringes are not possible to re-use.

Further, the invention has mainly been described with reference to a few embodiments. However, as is readily understood by a person skilled in the art, other embodiments than the ones disclosed above are equally possible within the scope of the invention, as defined by the appended claims.

In the claims, the term “comprises/comprising” does not exclude the presence of other elements or steps. Furthermore, although individually listed, a plurality of means, elements or method steps may be implemented by e.g. a single unit or processor. Additionally, although individual features may be included in different claims, these may possibly advantageously be combined, and the inclusion in different claims does not imply that a combination of features is not feasible and/or advantageous. In addition, singular references do not exclude a plurality. The terms “a”, “an”, “first”, “second” etc do not preclude a plurality. Reference signs in the claims are provided merely as a clarifying example and shall not be construed as limiting the scope of the claims in any way.

1. A plunger rod for use in connection with an injection syringe barrel, the plunger rod comprising:
an elongated rod having
a proximal end adapted to receive an applied force along an axis, and
a distal end adapted to transmit the, applied force along the axis;
wherein the plunger rod is adapted to be inserted into a barrel of a syringe extending along the axis, the plunger rod being adapted to transmit force along the axis to a plunger stopper disposed within the barrel; and
a radial spring mechanism provided the elongated rod, the radial spring mechanism adapted to interact with an inner surface of the barrel.

2. The plunger rod according to claim 1, wherein the plunger rod is made of a thermoplastic polymer having a Young's modulus within the interval 500 MPa to 2000 MPa.

3. The plunger rod according to claim 1, wherein the elongated rod has a cross-shaped cross-section comprising four supporting ribs extending radially from a center of the elongated rod, and
wherein at least one of the supporting ribs includes the radial spring mechanism.

4. The plunger rod according to claim 3, wherein the four supporting ribs include interaction knobs.

5. The plunger rod according to claim 3, wherein the four supporting ribs include interaction discs.

6. The plunger rod according to claim 3, wherein saiddie distal end of the elongated rod includes a spring portion adapted to be displaced radially from a relaxed position once the plunger rod is inserted into the barrel of the syringe.

7. The plunger rod according to claim 6, wherein at least one of the four supporting ribs includes the spring portions.

8. The plunger rod according to claim 7, wherein the spring portion includes an axial slit.

9. The plunger rod according to claim 8, wherein the axial slit is arranged in a middle of the elongated rod.

10. The plunger rod according to claim 8, wherein the axial slit is contained within the at least one supporting rib (115).

11. The plunger rod according to claim 7, wherein the spring portion includes an interaction spoke.

12. The plunger rod according to claim 7, wherein the spring portion includes a set of reeds.

13. A syringe comprising:
a barrel with a needle arranged at a distal end of the barrel, the barrel being adapted to contain a drug, the barrel having a tubular wall extending proximally from the distal end, whereby the barrel extends along an axis;
a plunger stopper disposed within the barrel, the plunger stopper being adapted to be in fluid-tight engagement with the tubular wall of the barrel, the plunger stopper being displaceable along the axis within the barrel;
wherein the barrel includes an opening at a proximal end of the barrel; and
a plunger rod according to claim 1 extending along the axis;
wherein the plunger stopper is adapted to receive force along the axis from the plunger rod.

14. The syringe according to claim 13, wherein the syringe contains the drug.

15. The syringe according to claim 13, wherein the plunger stopper is made of an elastomer having a Young's modulus within the interval 25 MPa to 300 MPa.

16. The syringe according to claim 13, wherein the plunger stopper is adapted to only be displaceable along the axis towards the distal end of the barrel.

17. The syringe according to claim 13, wherein the plunger stopper includes a thread profile.
